(19) United States
(12) Patent Application Publication
(10) Pub. No.: US 2023/0193204 A1
(43) Pub. Date: Jun. 22, 2023

(54) TREATMENT OF ERECTILE DYSFUNCTION BY FIBROBLAST ADMINISTRATION

(71) Applicant: FIGENE, LLC, Houston, TX (US)

(72) Inventor: Thomas ICHIM, San Diego, CA (US)

(21) Appl. No.: 17/996,033

(22) PCT Filed: Apr. 10, 2021
(86) PCT No.: PCT/US2021/026752

§ 371 Date: Oct. 12, 2022

Related U.S. Application Data
(60) Provisional application No. 63/008,970, filed on Apr. 13, 2020.

(51) Int. Cl. C12N 5/077; A61P 15/10; C12N 15/63

(52) U.S. Cl. CPC C12N 5/0656; A61P 15/10; C12N 15/63; C12N 2500/02

(57) ABSTRACT

In some aspects, disclosed herein are methods and compositions for treatment of erectile dysfunction using regenerative fibroblasts or cells derived from fibroblasts. Methods and compositions disclosed also include those for generating regenerative fibroblast cells from fibroblasts. Also disclosed are methods and compositions for treatment of erectile dysfunction using regenerative fibroblast-conditioned media. Regenerative fibroblast cells generated from fibroblasts and regenerative fibroblast-conditioned media are also described.

This application claims priority to U.S. Provisional Patent Application Ser. No. 63/008,970, filed Apr. 13, 2020, which is incorporated by reference herein in its entirety.

### TECHNICAL FIELD

Embodiments of the disclosure generally include at least the fields of cell biology, molecular biology, urology, and medicine. More particularly, the disclosure pertains to the area of erectile dysfunction and the use of fibroblasts for treatment of erectile dysfunction.

### BACKGROUND

Erectile dysfunction (ED) is characterized by the lack of ability to achieve and maintain penile erection for intercourse. In our aging society, ED is becoming an increasing problem. According to one study 39% of men at age 40 experience symptoms of ED, whereas by age 70 the incidence rises to 67%. It is estimated that 10-30 million Americans suffer from this condition. In addition to aging it is believed that 50-85% of ED cases are associated with conditions that affect the endothelium such as hypertension, diabetes, cardiovascular disease, and dyslipidemia.

Erections are achieved and maintained by the erectile bodies of the penis. The penis is comprised of three erectile bodies, two which are parallel termed corpora cavernosa, and underneath, wedged in between, the corpus spongiosum, which contains the urethra. The three erectile bodies are heavily vascularized and contain a large proportion of smooth muscle cells. Erection is caused by neurologically-induced relaxation of smooth muscle cells in the erectile bodies, which allows influx and accumulation of blood into balloon-like sacs between the smooth muscle cells called sinusoids. As blood accumulates, outflow of blood is prevented by pressure from the tunica albuginea against the venous plexus, thus trapping the blood and allowing erection to occur. The process of blood accumulation due to venous trapping is termed the veno-occlusive mechanism. Additional rigidity of the penis shaft is provided by contraction of the ischiocaverous muscles.

Initial nerve impulses triggering erections originate in the brain in response to sexual stimuli. Although this area is currently under investigation, certain parts of the brain have been found to be involved in arousal. These include structures such as the frontal lobe, hypothalamus, thalamus, amygdala, and cingulated gyrus. The first identification of a brain structure associated with erection occurred in the 1960s in primate experiments using brain electrostimulation via a stereotactic technique. These studies demonstrated that stimulation of the medial frontal lobe led to erections in a consistently reproducible manner. Subsequent clinical experiments demonstrated that during exposure to sexual stimuli, functional MRI accurately identified increased perfusion activity in the frontal lobe, the cingulated gyrus, and the thalamus. Interventional evidence of the importance of distinct anatomical areas in the central control of erection also comes from clinical studies showing deep brain stimulation of the thalamic area for treatment of Tourette's syndrome leads to unexpected erections as a consequence. Furthermore, the successful use of centrally-acting drugs such as apomorphine in the treatment of ED supports the importance of cerebral control of erection. Additionally, older studies in pedophiles and rapists reported some success with central inhibition of abnormal sex drive through surgical ablation of certain anatomical regions of the hypothalamus.

In contrast to central regulation of erections, much more is known about spinal control. The sacral parasympathetic nucleus, the thoracolumbar sympathetic nuclei and the pudendal motoneurons are all involved in the stepwise transmission of signals between the dorsal penile nerves and central nervous system. Studies which demonstrated that electrostimulation of the roots of S2 to S55 in men with complete thoracic spinal injuries was able to produce erection in all patients assessed allowed identification of specific areas in the spinal cord responsible for erectile regulation. A variety of electro-spinal stimulation of erection studies have also determined that during normal erection, parasympathetic nervous activation causes relaxation of smooth muscle and dilation of the helicine arteries in the corpora cavernosum and the corpus spongiosum. This dilation, in combination with the veno-occlusive mechanism which prevents blood outflow from the penile body, causes the end result of erection. Mechanistically, parasympathetic activation causes upregulation of nitric oxide (NO) production by nonadrenergic and noncholinergic nerves, as well as the endothelium which lines the penile arteries and cavernosal sinusoids. Accumulation of NO increases production of cyclic guanosine monophosphate (cGMP) through activation of the enzyme guanylyl cyclase. cGMP acts as a second messenger which leads to decreased calcium uptake into the endothelial-lined smooth muscles of the cavernosal sinusoids, thus causing relaxation and erection. Since phosphodiesterase (PDE)-5 is involved in the breakdown of cGMP, the inhibition of PDE-5 has been chosen as a pharmacological goal of medications such as Viagra (sildenafil), Cialis (tadalafil), and Levitra (vardenafil).

Recognition by practitioners of the broad spectrum of diagnostic tools for diagnosing ED is important in deciding which is best for respective patient populations. ED is primarily diagnosed clinically based on symptomology. First assessments of ED typically include physical examination with special attention to possible penile and scrotal pathology or abnormalities, which may alter or inhibit the ability of the penis to cause penetration. Anatomical abnormalities or visible injuries are usually a small percentage of causative factors in ED, however. After physical examination, more detailed tests are performed, such as the penobrachial blood pressure index (PBPI), doppler investigation of the penile arteries, and the papaverine test. These are described in detail in U.S. Pat. No. 6,132,757, incorporated by reference herein.

The PBPI is the penile systolic blood pressure divided by the systolic blood pressure determined at one of the arms of the patient. These blood pressures can be determined by any number of standard techniques. Thus, the penile systolic blood pressure can be determined by i) placing an inflatable cuff capable of applying variable pressure, readable from a gauge, around the base of the free part of the penis in the flaccid state; ii) localizing the penile arteries with a Doppler ultrasound probe (e.g., an 8 MHz probe, such as the Mini Doplex D500, available from Huntleigh Technology, Luton, United Kingdom); and iii) inflating and deflating the cuff to ascertain the pressure at which the Doppler sound reappears. The pressure at which the Doppler sound reappears is the penile systolic blood pressure. Normal male penile blood pressure is reflected by a PBPI is >0.80. With regard to Doppler investigation, each of the two penile cavernous arteries is investigated distal to the aforementioned cuff using the Doppler ultrasound problem. The function of each of the two arteries is assessed by Doppler ultrasound using an arbitrary scale of 0, 1, 2 or 3, where 0 means that the function is so deficient that the artery cannot be located and 3 means that the artery is well enough that maximal Doppler sound is observed. Doppler analysis and papaverine tests have been thoroughly characterized in prior studies.

Other methods used to quantify ED include the International Index of Erectile Function (IIEF) and the Erectile Function Visual Analog Scale (EF-VAS). EF-VAS has become the gold standard for diagnosis and evaluation of treatment efficacy since it is based on overall functionality rather than specific anatomical or biological abnormalities/defects.

The current standard of care for treatment of ED is phosphodiesterase inhibitors. For example, U.S. Pat. No. 5,250,534 discloses sildenafil (VIAGRA), an orally available PDE5 inhibitor. Additional PDE5 inhibitors include Cialis (tadalafil), Stendra (avanafil), and Levitra (vardenafil). These inhibitors are used to treat various forms of ED, including organic and psychogenic ED as well as ED caused by concurrent diabetes, hypertension, hypercholesterolemia, cardiovascular disease, and/or a history of urologic pelvic surgery. PDE5 inhibitors effect treatment of ED by augmenting the physiologic process of tumescence. During penile erection, nitric oxide stimulates cyclic guanosine monophosphate (cGMP) production in vascular smooth muscle cells of the corpus cavernosum. cGMP acts through a series of intracellular pathways to lower the intracellular level of calcium which, in turn, causes cavernosal smooth muscle relaxation and penile arterial vessel dilation. PDE5 inhibitors generally work to decrease cGMP metabolism for successful attainment and maintenance of an erection.

Unfortunately, up to 50% of patients are unresponsive to PDE5 inhibitor therapy or do not tolerate adverse effects associated with treatment. Major factors associated with resistance to the effects of PDE5 inhibitors in substantial numbers of patients include atherosclerosis, vascular disease, diabetes mellitus, nerve damage or other advanced neurologic damage, and smooth muscle atrophy. Adverse effects associated with PDE5 inhibitors include optic neuropathy, headaches, and various cardiovascular pathologies, especially when taken in combination with nitrate medications. In fact, in 1998, the US Food and Drug Administration published a report on 130 confirmed deaths among men who received prescriptions for sildenafil citrate, where causes of death included arrythmias, sudden cardiac death and hypotension-associated events. In addition, PDE5 inhibitors are known to possess a variety of systemic effects in numerous organ systems, causing uncertainty regarding the long-term effects of PDE5 inhibition.

Several approaches have demonstrated promise in the improvement of responsiveness to PDE5 inhibitors, including administration of propionyl-L-carnitine, intracavernous PGE1, and testosterone gel. Beneficial non-ED uses of PDE5 inhibitors are also known. For example, investigators hypothesized and demonstrated inhibition of symptomatic pulmonary arterial hypertension in a double blind clinical trial after administration of sildenafil citrate upon inhibition of PDE5 expressed in lung tissue. However, given that PDE5 is expressed throughout the body, including expression in platelets, the kidneys, and the pancreas, prolonged systemic inhibition of this enzymatic system may have adverse physiologic consequences.

An additional pharmacological method to treat ED includes intracavernosal and/or intra-urethral administration of vasodilators. For example, MUSE (U.S. Pat. No. 5,773,020) is a single-use, medicated transurethral micro-suppository for the delivery of alprostadil, a vasodilator, to the male urethra indicated for the treatment of ED. However, serious adverse reactions to a single therapeutic dose of PGE1, the active ingredient in MUSE, include pain and priapism in about 3 to 10 percent of patients, which increases with continued use. Additionally, manipulation of the penis to insert the micro-suppository prior to intercourse is not desirable by many patients. Intracavernosal injections of vasoactive drugs, such as CAVERJECT (U.S. Pat. No. 4,127,118) are satisfactory or effective in 30 to 90 percent of men, but they can be associated with pain, priapism, penile hematomas, cutaneous ecchymosis, corporal fibrosis, and in some situations damage to the penile nerves. Devices for intrascrotal implantation have been described in U.S. Pat. No. 5,518,499, which allow for administration of vasoactive agents such as PGE1 without the need for intracavernosal injections. However this approach is highly invasive and does not treat physiological causes of ED.

Other pharmacological treatment methods that have been patented but have not been clinically approved include: a) U.S. Pat. No. 3,943,246, which describes treatment of impotence administration of oxytocin; b) U.S. Pat. No. 4,530,920, which describes nonapeptide and decapeptide analogs of luteinizing hormone releasing hormone agonists for treatment of ED; c) U.S. Pat. No. 4,139,617, which describes 19-oxygenated-androst-5-enes treatment of ED; and d) U.S. Pat. No. 5,541,211, which describes the use of yohimbine in treatment of ED. Centrally acting treatments for erectile dysfunction include apomorphine, as disclosed in U.S. Pat. No. 5,770,606. U.S. Pat. No. 4,801,587 discloses that phentolamine (VASOMAX™) which is available in a number of countries for treating hypertension is also useful for treating ED.

Non-pharmacological treatments for ED are increasingly invasive and include vacuum pumps, penile prostheses, and vascular surgery. Vacuum pumps may be difficult for some men to use, do not allow for spontaneous, natural erections to occur, and may cause penile trauma if used improperly. Implantation of penile prostheses, described in U.S. Pat. No. 5,065,744, is invasive, expensive, irreversible, and can cause penile deformity. Clinical interest in penile revascularization surgery stems from the widely reported link between ED and atherosclerotic vascular disease. Unfortunately, however, the success rate of vascular surgery has been reported to be highly variable, which has raised questions concerning the safety and feasibility of stent-based therapies and the appropriate means for diagnosis of arteriogenic ED. Hence, there is clearly a need for additional treatments to address ED.

As an alternative to invasive procedures or oral phosphodiesterase inhibitors, administration of agents capable of inducing endothelial cell proliferation and/or neoangiogenesis have been shown to induce inhibition of ED progression or reversion of ED in animal models. For example, basic fibroblast growth factor (bFGF) is a known inducer of angiogenesis in ischemic situations, and its exogenous administration is therapeutic in models of stroke, angina, and peripheral limb ischemia. The administration of two 2.5 microgram doses of bFGF, separated by a 3-week interval, into the corporal tissue of hypercholesterolemic rabbits was shown to increase corporal relaxation in response to chemical stimuli and the ability to generate NO. Other studies in hypercholesterolemic rabbits have demonstrated that systemic basic fibroblast growth factor induces favorable histological changes in the corpus cavernosum. Finally, in another study, intracavernous administration of bFGF into diabetic rats by means of gelatin microbeads resulted in protection of erectile function from diabetes mediated onset of ED.

Inhibition of PDE5 upregulates erectile mechanisms, such as potentiating the effects of NO, but producing an erection still depends on the presence of substantial concentrations of functional erectile tissue. For example, a high adipose-to-smooth muscle ratio in cavernous tissue in conjunction with decreased expression or activity of neuronal or endothelial NO synthase (NOS), impaired NO release, accelerated NO destruction, and atrophy of cavernosal structures is associated with resistance to PDE5 inhibitors in ED patients. Accordingly, instead of augmenting the activity of the already diminished tissue in patients with ED, there is need for a means of regenerating erectile tissue or components thereof in a natural and physiological manner to treat ED.

The present disclosure provides solutions to long-felt needs in the art of erectile dysfunction.

### BRIEF SUMMARY

The present disclosure, in at least some embodiments, is directed to methods and compositions related to treating or preventing ED, including delaying the onset and/or reducing the severity and/or reducing the risk of, ED, including complete inhibition of ED. Disclosed herein are methods and compositions for administration of fibroblast cells able to inhibit progression of one or more pathological processes associated with ED and that are able to induce regeneration of erectile tissue or components thereof to ameliorate and/or reverse the pathological processes. In particular embodiments, compositions of the present disclosure comprise cells cultured in vitro under specific culture conditions and/or growth factors sufficient to enhance the ability of the cells to inhibit neuronal cell dysfunction, inhibit cavernosal fibrosis, inhibit smooth muscle degeneration, inhibit biological pathways causative of ischemia, or a combination thereof. In particular embodiments, these cells comprise at least fibroblasts.

In some embodiments of the present disclosure, the method comprises treating or preventing ED, including delaying the onset of and/or reducing the severity of and/or reducing the risk of, ED, including complete inhibition of ED, in an individual, comprising administering a therapeutically effective amount of a composition comprising fibroblasts and/or conditioned media therefrom to an individual in need thereof. In some embodiments of the present disclosure, the method comprises generating regenerative fibroblast cells from fibroblasts, comprising subjecting fibroblasts to one or more conditions sufficient to generate regenerative fibroblast cells from the fibroblasts. In some embodiments of the present disclosure, the composition comprises regenerative fibroblast cells, wherein the regenerative fibroblast cells are derived from a fibroblast cultured under one or more conditions sufficient to enhance the ability of the regenerative fibroblast cells to induce angiogenesis, prevent tissue atrophy, regenerate functional tissue, inhibit neuronal cell dysfunction, inhibit cavernosal fibrosis, inhibit smooth muscle degeneration, inhibit biological pathways causative of ischemia, or a combination thereof. In some embodiments of the present disclosure, the composition comprises regenerative fibroblast-conditioned media for ED treatment wherein the media is derived from regenerative fibroblast cells cultured under conditions sufficient to upregulate production of one or more growth factors.

In some embodiments, the fibroblasts comprise regenerative fibroblasts. In some embodiments, the regenerative fibroblast cells comprise one or more of the following biological activities: (a) induction of angiogenesis; (b) prevention of tissue atrophy;(c) regeneration of functional tissue; (d) inhibition of neuronal cell dysfunction; (e) inhibition of cavernosal fibrosis; (f) inhibition of smooth muscle degeneration; and (g) inhibition of one or more biological pathways causative of ischemia.

In some embodiments, the fibroblast cells are cultured under conditions sufficient to differentiate the fibroblasts into regenerative fibroblast cells. In some embodiments, the regenerative fibroblast cells are cultured under conditions sufficient to enhance the ability of the regenerative fibroblast cells to induce angiogenesis, prevent tissue atrophy, regenerate functional tissue, inhibit neuronal cell dysfunction, inhibit cavernosal fibrosis, inhibit smooth muscle degeneration, inhibit biological pathways causative of ischemia, or a combination thereof. In some embodiments, the conditions comprise hypoxia. In some embodiments, the hypoxic conditions comprise from 0.1% oxygen to 10% oxygen, and any range derivable therein, for a period of 30 minutes to 3 days and any range derivable therein. In some embodiments, the hypoxic conditions comprise 3% oxygen for 1-24 hours, and any range derivable therein. In some embodiments, hypoxic conditions are chemically induced. In some embodiments, chemical induction of hypoxia comprises culture in a sufficient amount of cobalt (II) chloride. In some embodiments, fibroblast cells are cultured with 1 μM-300 μM cobalt (II) chloride and any range derivable therein. In some embodiments, the fibroblast cells are incubated with 250 μM of cobalt (II) chloride. In some embodiments, the fibroblast cells are further cultured for 1-48 hours, and any range derivable therein. In some embodiments, the fibroblast cells are cultured for a time period of 1-24 hours and any range derivable therein. In some embodiments, the hypoxic conditions induce upregulation of HIF-1α. In some embodiments, expression of HIF-1α is detected by expression of VEGF secretion. In some embodiments, the hypoxic conditions induce upregulation of CXCR4 on the fibroblast cells. In some embodiments, upregulation of CXCR4 promotes homing of the fibroblast cells to an SDF-1 gradient. In some embodiments, the conditions further comprise treatment of the regenerative fibroblast cells with one or more growth factors, one or more differentiation factors, one or more dedifferentiation factors, or a combination thereof.

In some embodiments, the regenerative fibroblast cells express one or more markers selected from the group consisting of Oct-4, Nanog, Sox-2, KLF4, c-Myc, Rex-1, GDF-3, LIF receptor, CD105, CD117, CD344, Stella, and a combination thereof. In some embodiments, the regenerative fibroblast cells do not express one or more cell surface proteins selected from the group consisting of MHC class I, MHC class II, CD45, CD13, CD49c, CD66b, CD73, CD105, CD90, and a combination thereof. In some embodiments, the regenerative fibroblast cells have enhanced GDF-11 expression compared to a control or standard.

In some embodiments, the fibroblast cells are, or are derived from, fibroblasts isolated from umbilical cord, skin, cord blood, adipose tissue, hair follicle, omentum, bone marrow, peripheral blood, Wharton's Jelly, or a combination thereof. In some embodiments, the fibroblast cells are obtained from dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof. In some embodiments, the fibroblast cells are autologous, allogeneic, or xenogeneic to the recipient. In some embodiments, the fibroblast cells are purified from bone marrow. In some embodiments, the fibroblast cells are purified from peripheral blood. In some embodiments, the regenerative fibroblast cells are isolated from peripheral blood of an individual who has been exposed to one or more conditions and/or one or more therapies sufficient to stimulate regenerative fibroblast cells from the individual to enter the peripheral blood of the individual. In some embodiments, the conditions sufficient to stimulate regenerative fibroblast cells from the individual to enter the peripheral blood comprise administration of an effective amount of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. In some embodiments, the therapies sufficient to stimulate regenerative fibroblast cells from the individual to enter the peripheral blood comprise therapies including exercise, hyperbaric oxygen, autohemotherapy by ex vivo ozonation of peripheral blood, induction of SDF-1 secretion in an anatomical area outside of the bone marrow, or a combination thereof.

In some embodiments, the regenerative fibroblast cells are comprised of an enriched population of regenerative fibroblast cells. In some embodiments, enrichment is achieved by: (a) transfecting the cells with a suitable vector (including viral (adenoviral, retroviral, lentiviral, or adeno-associated viral) or non-viral (such as a plasmid or transposon)) comprising a fibroblast-specific promoter operably linked to a reporter or selection gene, wherein the reporter or selection gene is expressed, and (b) enriching the population of cells for cells expressing the reporter or selection gene. In some embodiments, enrichment is achieved by: (a) treating the cells with a detectable compound, wherein the detectable compound is selectively detectable in proliferating and non-proliferating cells, and (b) enriching the population of cells for proliferating cells. In some embodiments, the detectable compound is selected from the group consisting of carboxyfluorescein diacetate, succinimidyl ester, Aldefluor, and a combination thereof.

In some embodiments, the regenerative fibroblast cells are reprogrammed fibroblasts. In some embodiments, the reprogrammed fibroblasts are selected from the group consisting of cells subsequent to a nuclear transfer, cells subsequent to a cytoplasmic transfer, cells treated with one or more DNA methyltransferase inhibitors, cells treated with one or more histone deacetylase inhibitors, cells treated with one or more GSK-3 inhibitors, cells induced to dedifferentiate by alteration of one or more extracellular conditions, and cells exposed to various combinations of treatment conditions. In some embodiments, the DNA methyltransferase inhibitor is selected from the group consisting of 5-azacytidine, psammaplin A, zebularine, and a combination thereof. In some embodiments, the DNA histone deacetylase inhibitor is selected from the group consisting of valproic acid, trichostatin-A, trapoxin A, depsipeptide, and a combination thereof.

In some embodiments, the regenerative fibroblast cells are fibroblasts isolated as side population cells. In some embodiments, the fibroblasts isolated as side population cells are identified based on expression of the multidrug resistance transport protein (ABCG2). In some embodiments, the fibroblasts isolated as side population cells are identified based on the ability to efflux intracellular dyes. In some embodiments, the side population cells are derived from tissues selected from the group consisting of pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and a combination thereof.

In some embodiments, a committed fibroblast progenitor cell population is administered together with the fibroblast cells. In some embodiments, the committed fibroblast progenitor cells are selected from the group consisting of committed endothelial progenitor cells, committed neuronal progenitor cells, committed hematopoietic progenitor cells, and a combination thereof. In some embodiments, a committed endothelial progenitor cell population is administered in combination with the fibroblast cells. In some embodiments, the committed endothelial progenitor cells express one or more markers selected from the group consisting of CD31, CD34, AC133, CD146, flk1, and a combination thereof. In some embodiments, the committed endothelial progenitor cells are autologous, allogeneic, or xenogeneic to the recipient. In some embodiments, the committed endothelial progenitor cells are obtained from bone marrow. In some embodiments, the committed endothelial progenitor cells are obtained from peripheral blood. In some embodiments, committed endothelial progenitor cells are isolated from peripheral blood of an individual who has been exposed to one or more conditions and/or one or more therapies sufficient to stimulate endothelial progenitor cells from the individual to enter the peripheral blood of the individual. In some embodiments, the one or more conditions sufficient to stimulate committed endothelial progenitor cells from the individual to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. In some embodiments, the one or more therapies sufficient to stimulate committed endothelial progenitor cells from the individual to enter the peripheral blood comprise therapies including exercise, hyperbaric oxygen, autohemotherapy by ex vivo ozonation of peripheral blood, induction of SDF-1 secretion in an anatomical area outside of the bone marrow, or a combination thereof.

In some embodiments, testosterone is also administered to the individual. In some embodiments, the concentration of testosterone is sufficient to induce smooth muscle cell growth. In some embodiments, the concentration of testosterone is sufficient to induce migration of endothelial progenitor cells to the penis and/or upstream of the penis. In some embodiments, the testosterone is administered systemically or locally to the individual. In some embodiments, local administration of testosterone is by topical application, urethral suppository, and/or intracavernous injection.

In some embodiments, a therapeutically effective amount of one or more antioxidants are administered to the individual. In some embodiments, the one or more antioxidants are selected from the group consisting of ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, and a combination thereof. In some embodiments, the antioxidant is administered prior to administration of the fibroblast cells at a concentration sufficient to reduce oxidative stress. In some embodiments, the antioxidant is administered concurrently with the fibroblast cells at a concentration sufficient to reduce oxidative stress. In some embodiments, the antioxidant is administered subsequent to the fibroblast cells at a concentration sufficient to reduce oxidative stress.

In some embodiments, the erectile dysfunction comprises vascular insufficiency. In some embodiments, the erectile dysfunction comprises neuronal dysfunction. In some embodiments, the erectile dysfunction comprises fibrosis of the corpora cavernous, the corpus spongiosum, or a combination thereof. In some embodiments, the erectile dysfunction is associated with injury. In some embodiments, the injury is traumatic. In some embodiments, the injury is surgical. In some embodiments, the injury is atherosclerotic. In some embodiments, the injury is due to age-associated degeneration of neurons, smooth muscle, or a combination thereof. In some embodiments, structural elements of the neurons, smooth muscle, or a combination thereof degenerate. In some embodiments, structural elements include cellular morphology, cytoskeleton shape, and subcellular organelles. In some embodiments, functional elements of the neurons, smooth muscle, or a combination thereof degenerate. In some embodiments, functional elements include the ability of neurons to produce and respond to neurotransmitters and/or the ability of in the cause of smooth muscle to contract upon receiving contractile signals.

In some embodiments, the method is further defined as administering to the individual a therapeutically effective amount of a composition comprising regenerative fibroblast-conditioned media. In some embodiments, regenerative fibroblast cells are cultured under conditions sufficient to upregulate production of one or more growth factors in the regenerative fibroblast-conditioned media. In some embodiments, the conditions comprise hypoxia, hyperthermia, treatment with histone deacetylase inhibitors, or a combination thereof. In some embodiments, the regenerative fibroblast-conditioned media is concentrated. In some embodiments, the regenerative fibroblast-conditioned media is administered locally or systemically to the individual.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### DETAILED DESCRIPTION

### I. Examples of Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

The terms “reduce,” “inhibit,” “diminish,” “suppress,” “decrease,” “prevent” and grammatical equivalents (including “lower,” “smaller,” etc.) when in reference to the expression of any symptom in an untreated subject relative to a treated subject, mean that the quantity and/or magnitude of the symptoms in the treated subject is lower than in the untreated subject by any amount that is recognized as clinically relevant by any medically trained personnel. In one embodiment, the quantity and/or magnitude of the symptoms in the treated subject is at least 10% lower than, at least 25% lower than, at least 50% lower than, at least 75% lower than, and/or at least 90% lower than the quantity and/or magnitude of the symptoms in the untreated subject.

As used herein, the term “therapeutically effective amount” is synonymous with “effective amount”, “therapeutically effective dose”, and/or “effective dose” refers to an amount of an agent sufficient to ameliorate at least one symptom, behavior or event, associated with a pathological, abnormal or otherwise undesirable condition, or an amount sufficient to prevent or lessen the probability that such a condition will occur or re-occur, or an amount sufficient to delay worsening of such a condition. As one example, an effective amount is the amount sufficient to ameliorate and/or reverse erectile dysfunction (ED) in an individual. In one example, an effective amount of cells is an amount of fibroblast cells effective to regenerate or repair erectile tissue or components thereof in an individual having or at risk for developing ED. In another example, an effective amount of cells is an amount of cells capable of inhibiting neuronal cell dysfunction, inhibiting cavernosal fibrosis, inhibiting smooth muscle degeneration, or inhibiting biological pathways causative of ischemia in an individual having ED. An effective amount of cells may be between 10³ and 10¹¹ cells. In some cases, an effective amount of cells is about 10⁴ cells. The appropriate effective amount to be administered for a particular application of the disclosed methods can be determined by those skilled in the art, using the guidance provided herein. For example, an effective amount can be extrapolated from in vitro and in vivo assays as described in the present specification. One skilled in the art will recognize that the condition of the individual can be monitored throughout the course of therapy and that the effective amount of a compound or composition disclosed herein that is administered can be adjusted accordingly.

“Erectile dysfunction” (ED) as used herein refers to the inability to achieve and maintain penile erection for sexual activity of any kind, including at least intercourse. Various forms of ED include organic and psychogenic ED, as well as ED caused by concurrent diabetes, hypertension, hypercholesterolemia, cardiovascular disease, vasectomy, and/or a history of urologic pelvic surgery, as examples. In addition to aging as a cause of ED, it is believed that 50-85% of ED cases are associated with conditions that affect the endothelium, such as hypertension, diabetes, cardiovascular disease, and dyslipidemia, as examples.

As used herein, the terms “treatment,” “treat,” or “treating” refers to intervention in an attempt to alter the natural course of the individual or cell being treated, and may be performed either for prophylaxis or during the course of pathology of a disease or condition. Treatment may serve to accomplish one or more of various desired outcomes, including, for example, preventing occurrence or recurrence of disease, alleviation of symptoms, and diminishment of any direct or indirect pathological consequences of the disease, preventing metastasis, lowering the rate of disease progression, amelioration or palliation of the disease state, and remission or improved prognosis.

As used herein, “cell culture” means conditions wherein cells are obtained (e.g., from an organism) and grown under controlled conditions (“cultured” or grown “in culture”) outside of an organism. A primary cell culture is a culture of cells taken directly from an organism (e.g., tissue cells, blood cells, cancer cells, neuronal cells, fibroblasts, etc.). Cells are expanded in culture when placed in a growth medium under conditions that facilitate cell growth and/or division. The term “growth medium” means a medium sufficient for culturing cells. Various growth media may be used for the purposes of the present disclosure including, for example, Dulbecco's Modified Eagle Media (also known as Dulbecco's Minimal Essential Media) (DMEM), or DMEM-low glucose (also DMEM-LG herein). DMEM-low glucose may be supplemented with fetal bovine serum (e.g., about 10% v/v, about 15% v/v, about 20% v/v, etc.), antibiotics, antimycotics (e.g., penicillin, streptomycin, and/or amphotericin B), and/or 2-mercaptoethanol. Other growth media and supplementations to growth media are capable of being varied by the skilled artisan. The term “standard growth conditions” refers to culturing cells at 37° C. in a standard humidified atmosphere comprising 5% CO₂. While such conditions are useful for culturing, it is to be understood that such conditions are capable of being varied by the skilled artisan who will appreciate the options available in the art for culturing cells. When cells are expanded in culture, the rate of cell proliferation is sometimes measured by the amount of time needed for the cells to double in number. This is referred to as doubling time.

“Differentiation” (e.g., cell differentiation) describes a process by which an unspecialized (or “uncommitted”) or less specialized cell acquires the features (e.g., gene expression, cell morphology, etc.) of a specialized cell, such as a nerve cell or a muscle cell for example. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. In some embodiments of the disclosure, “differentiation” of fibroblasts to neuronal cells is described. This process may also be referred to as “transdifferentiation”.

As used herein, “dedifferentiation” refers to the process by which a cell reverts to a less specialized (or less committed) position within the lineage of a cell. As used herein, the lineage of a cell defines the heredity of the cell. The lineage of a cell places the cell within a hereditary scheme of development and differentiation. Within the context of the current disclosure, “dedifferentiation” may refer to fibroblasts acquiring more “immature” associated markers such as OCT4, NANOG, RAS, RAF, CTCF-L, FLT3, and SOX2. Additionally, “dedifferentiation” may mean acquisition of functional properties such as enhanced proliferation activity and/or migration activity towards a chemotactic gradient. In some embodiments fibroblasts may be “dedifferentiated” by treatment with various conditions, subsequent to which they are “differentiated” into other cell types.

“Fibroblasts” include isolated fibroblast cells or population(s) thereof capable of proliferating and differentiating into ectoderm, mesoderm, or endoderm, wherein the isolated fibroblast cell expresses at least one of Oct-4, Nanog, Sox-2, KLF4, c-Myc, Rex-1, GDF-3, LIF receptor, CD105, CD117, CD344 or Stella markers, and does not express at least one of MHC class I, MHC class II, CD45, CD13, CD49c, CD66b, CD73, CD105, or CD90 cell surface proteins. These cells may be cultured in a growth medium to obtain conditioned media. These cells may also be cultured alone or may be cultured in the presence of other cells, including in order to further upregulate production of growth factors in the conditioned media.

As used herein, “regenerative” refers to the ability of fibroblasts of the present disclosure to effect therapeutic functions, production of growth factors, stimulation of angiogenesis, inhibition of inflammation, and/or augmentation of tissue self-renewal, for example in part through activation of endogenous and/or exogenous stem and/or progenitor cells. “Regenerative activities” include but are not limited to the promotion of angiogenesis, suppression of inflammation, and secretion of growth factors such as IGF-1, EGF-1, FGF-2, VEGF, and FGF-11. Fibroblasts having regenerative activities can be isolated for specific markers and subsequently transfected with genes capable of endowing various therapeutic functions. Genes useful for stimulation of regenerative activities including augmentation of hematopoietic activity include interleukin-12 and interleukin-23 to stimulate proliferation of hematopoietic stem cells, for example. Other useful genes include interleukin-35, wherein interleukin-35 transfection allows for generation of cells possessing anti-inflammatory and angiogenic T regulatory cell activity, said cells possessing T regulatory cell activities include cells expressing the transcription factor FoxP3.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

A variety of aspects of this disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range as if explicitly written out. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. When ranges are present, the ranges may include the range endpoints.

The term “subject,” as used herein, may be used interchangeably with the term “individual” and generally refers to an individual in need of a therapy. The subject can be a mammal, such as a human, dog, cat, horse, cow, pig or rodent. The subject can be a patient, e.g., have or be suspected of having or at risk for having a disease or medical condition. For subjects having or suspected of having a medical condition directly or indirectly associated with bone, the medical condition may be of one or more types. The subject may have a disease or be suspected of having the disease. The subject may be asymptomatic. The subject may be of any gender. The subject may be of a certain age, such as at least 15, 20, 25, 30, 35, 40, 45, 50, 55, 60, 65, 70, 75, 80, 85, 90, 95, or 100 or more.

### II. Generation of Regenerative Fibroblasts

Certain aspects of the present disclosure relate to the generation of regenerative fibroblast cells for treatment or prevention of ED in an individual. Regenerative fibroblast cells may be generated by culturing fibroblasts under sufficient conditions to generate a regenerative fibroblast cell. In some embodiments, conditions sufficient to generate regenerative fibroblasts include culture in hypoxic conditions, selection for rhodamine 123 efflux, and culture with one or more dedifferentiation agents such as one or more histone deacetylase inhibitors. In some embodiments, the fibroblast cells can provide a tissue with regenerative activity. In some embodiments, the method includes culturing the population of fibroblast regenerative cells under conditions that support proliferation of the cells. In other embodiments, the fibroblast cells may be cultured under conditions that form tissue aggregate bodies. In other embodiments, the regenerative fibroblasts are capable of inhibiting neuronal cell dysfunction, inhibiting cavernosal fibrosis, inhibiting smooth muscle degeneration, inhibiting biological pathways causative of ischemia, or a combination thereof.

Specific desirable properties of fibroblast cells of the present disclosure are the ability to increase endothelial function; induce neoangiogenesis; prevent atrophy; differentiate into functional penile tissue; and/or induce local resident stem and/or progenitor cells to proliferate through secretion of soluble factors or membrane bound activities. In one embodiment, fibroblast cells are collected from an autologous patient, expanded ex vivo, and reintroduced into the patient at a concentration and frequency sufficient to cause therapeutic benefit in ED. The fibroblast cells are selected for the ability to cause neoangiogenesis, prevent tissue atrophy, and regenerate functional tissue. In another embodiment, fibroblast cells are collected from an allogeneic individual, expanded ex vivo, and introduced into the patient that is not the allogeneic individual at a concentration and frequency sufficient to cause therapeutic benefit in ED. The fibroblast cells are selected for the ability to cause neoangiogenesis, prevent tissue atrophy, and regenerate functional tissue.

When selecting fibroblast cells, several factors must be taken into consideration, including the ability for ex vivo expansion without loss of therapeutic activity, ease of extraction, general potency of activity, and/or potential for adverse effects. Ex vivo expansion ability of fibroblasts can be measured using typical proliferation and colony assays known to one skilled in the art, while identification of therapeutic activity depends on functional assays that test biological activities such as the ability to support endothelial function, protect neurons from degeneration and/or atrophy, and/or inhibit smooth muscle atrophy and/or degeneration.

In some embodiments, assessment of therapeutic activity is performed using surrogate assays that detect one or more markers associated with a specific therapeutic activity. In some embodiments, assays used to identify therapeutic activity of fibroblast cell populations include evaluation of the production of one or more factors associated with desired therapeutic activity. In some embodiments, evaluation of the production of one or more factors to approximate therapeutic activity in vivo includes identification and quantification of the production of FGF, VEGF, angiopoietin, a combination thereof, or other angiogenic molecules that may be used to serve as a guide for approximating therapeutic activity in vivo. In specific embodiments, secretion of factors that inhibit smooth muscle atrophy or neuronal dysfunction are also used as markers for identification of cells that are useful for ED therapy.

In one embodiment, regenerative fibroblast cells are purified from cord blood. Cord blood fibroblast cells are fractionated, and the fraction with enhanced therapeutic activity is administered to the patient. In some embodiments, cells with therapeutic activity are enriched based on physical differences (e.g., size or density), electrical potential differences (e.g., membrane potential), differences in uptake or excretion of certain compounds (e.g., rhodamine 123 efflux), as well as differences in expression marker proteins (e.g., CD73). Distinct physical property differences between stem cells with high proliferative potential and low proliferative potential are known. Accordingly, in some embodiments, cord blood fibroblast cells with a higher proliferative ability are selected, whereas in other embodiments, a lower proliferative ability is desired. In some embodiments, cells are directly injected into the area of need, such as in the corpora cavernosa, and should be substantially differentiated. In other embodiments, cells are administered systemically and should be less differentiated, so as to still possess homing activity to the area of need.

In embodiments where specific cellular physical properties are the basis of differentiating between cord blood fibroblast cells with various biological activities, discrimination on the basis of physical properties can be performed using a Fluorescent Activated Cell Sorter (FACS), through manipulation of the forward scatter and side scatter settings. Other embodiments include methods of separating cells based on physical properties using filters with specific size ranges, density gradients, and pheresis techniques. In other embodiments where differentiation is based on electrical properties of cells, techniques such as electrophotoluminescence are used in combination with a cell sorting means such as FACS. In some embodiments, selection of cells is based on ability to uptake certain compounds as measured by the ALDESORT system, which provides a fluorescent-based means of purifying cells with high aldehyde dehydrogenase activity. Without being bound by theory, cells with high levels of this enzyme are known to possess higher proliferative and self-renewal activities in comparison to cells possessing lower levels. Other embodiments include methods of identifying cells with high proliferative activity by identifying cells with ability to selectively efflux certain dyes such as rhodamine-123, Hoechst 33342, or a combination thereof. Without being bound to theory, cells possessing this property often express the multidrug resistance transport protein ABCG2 and are known for enhanced regenerative ability compared to cells which do not possess this efflux mechanism.

In other embodiments, cord blood cells are purified for certain therapeutic properties based on the expression of one or more markers. In one particular embodiment, cord blood fibroblast are purified for cells with the endothelial precursor cell phenotype. Endothelial precursor cells or progenitor cells express markers such as CD133, CD34, or a combination thereof and are purified by positive or negative selection using techniques such as magnetic activated cell sorting (MACS), affinity columns, FACS, panning, other means known in the art, or a combination thereof. In some embodiments, cord blood-derived endothelial progenitor cells are administered directly into the target tissue for ED, while in other embodiments, the cells are administered systemically. In some embodiments, the endothelial precursor cells are differentiated in vitro and infused into a patient. Verification of endothelial differentiation is performed by assessing ability of cells to bind FITC-labeled Ulex europaeus agglutinin-1, ability to endocytose acetylated Di-LDL, and the expression of endothelial cell markers such as PECAM-1, VEGFR-2, or CD31.

In some embodiments, cord blood fibroblast cells are endowed with desired activities prior to administration into the patient. In one specific embodiment, cord blood cells are “activated” ex vivo by brief culture in hypoxic conditions to upregulate nuclear translocation of the HIF-1 transcription factor and endow the cord blood cells with enhanced angiogenic potential. In some embodiments, hypoxia is achieved by culture of cells in conditions of 0.1% oxygen to 10% oxygen, including 0.1%-7%, 0.1%-5%, 0.1%-2%, 0.1%-1%, 0.5%-10%, 0.5%-7%, 0.5%-5%, 0.5%-1%, 1%-10%, 1%-7%, 1%-5%, 2%-10%, 2%-7%, 2%-5%, or 5%-10%. In other embodiments, hypoxia is achieved by culture of cells in conditions of 0.5% oxygen and 5% oxygen. In other embodiments, hypoxia is achieved by culture of cells in conditions of about 0.1%, 0.5%, 1%, 2%, 3%, 4%, 5%, 6%, 7%, 8%, 9%, or 10% oxygen. Cells may be cultured for a variety of time points ranging from 1-72 (including 1-60, 1-48, 1-36, 1-24, 1-12, 12-72, 12-60, 12-48, 12-36, 12-24, 24-72, 24-60, 24-48, 24-36, 36-72, 36-60, 36-48, 48-72, 48-60, and 60-72) hours in some embodiments, including 13-59 hours in other embodiments and around 12, 24, 36, or 48 hours in still other embodiments. In one embodiment, cord blood cells are assessed for angiogenic or other desired activities prior to administration of the cord blood cells into the patient. Assessment methods are known in the art and include measurement of angiogenic factors, the ability to support viability and activity of cells associated with erectile function, and the ability to induce regeneration of the cellular components associated with erectile function.

In other embodiments, cord blood fibroblast cells are endowed with additional therapeutic properties through treatment ex vivo with factors such as de-differentiating compounds, proliferation-inducing compounds, compounds known to endow and/or enhance cord blood cells with useful properties, or a combination thereof. In one embodiment, cord blood cells are cultured with an inhibitor of the enzyme GSK-3 to enhance expansion of cells with pluripotent characteristics while maintaining the rate of differentiation. In another embodiment, cord blood cells are cultured in the presence of a DNA methyltransferase inhibitor such as 5-azacytidine to confer a “de-differentiation” effect. In another embodiment cord blood fibroblast cells are cultured in the presence of a differentiation agent that induces the cord blood stem cells to generate enhanced numbers of cells useful for treatment of ED after the cord blood cells are administered to a patient. For example, cord blood cells may be cultured in testosterone for a brief period such that subsequent to administration, an increased number of cavernosal smooth muscle cells are generated in a patient in need thereof.

In one embodiment, regenerative fibroblasts are purified from placental tissues. In contrast to cord blood fibroblast cells, in some embodiments, placental fibroblast cells are purified directly from placental tissues including the chorion, amnion, and villous stroma. In another embodiment, placental tissue is mechanically degraded in a sterile manner and treated with enzymes to allow dissociation of the cells from the extracellular matrix. Such enzymes include but are not restricted to trypsin, chymotrypsin, collagenases, elastase, hyaluronidase, or a combination thereof. In some embodiments, placental cell suspensions are subsequently washed, assessed for viability, and used directly by administration locally or systemically. In other embodiments, placental cell suspensions are purified to obtain certain populations with increased biological activity.

Purification may be performed using means known in the art including those used for purification of cord blood fibroblast cells. In some embodiments, purification may be achieved by positive selection for cell markers including SSEA3, SSEA4, TRA1-60, TRA1-81, c-kit, and/or Thy-1. In some embodiments, cells are expanded before introduction into the human body. Expansion can be performed by culture ex vivo with specific growth factors. Embodiments described for cord blood and embryonic stem also apply to placental stem cells.

In some embodiments, the regenerative fibroblast cells used for treatment or prevention of ED are capable of proliferating and differentiating into ectoderm, mesoderm, or endoderm. In some embodiments, the enriched population of fibroblast cells are about 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 micrometers in size. In some embodiments, fibroblast cells express a marker selected from the group consisting of Oct-4, Nanog, Sox-2, KLF4, c-Myc, Rex-1, GDF-3, LIF receptor, CD105, CD117, CD344, Stella, CD10, CD13, CD44, CD73, CD90, CD141, PDGFr-alpha, HLA-A, HLA-B, HLA-C, and a combination thereof. In some embodiments, fibroblast cells do not express MHC class I, MHC class II, CD45, CD13, CD49c, CD66b, CD73, CD105, CD90, CD31, CD34, CD45, CD117, CD141, HLA-DR, HLA-DP, HLA-DQ, or a combination thereof. In some embodiments, the method optionally includes the step of depleting cells expressing stem cell surface markers or MHC proteins from the cell population, thereby isolating a population of stem cells. In some embodiments, the cells to be depleted express MHC class I, CD66b, glycophorin a, and/or glycophorin b. In further embodiments, the fibroblast regenerative cell has enhanced expression of GDF-11 as compared to a control.

In some embodiments, fibroblasts are cultured under hypoxic conditions prior to administration in order to confer enhanced cytokine production properties and stimulate migration toward chemotactic gradients. Without wishing to be bound theory, protocols to enhance the regenerative potential of non-fibroblast cells using hypoxia can be modified or adapted for use with fibroblasts. For example, in one study, short-term exposure of MSCs to 1% oxygen increased mRNA and protein expression of the chemokine receptors CX3CR1 and CXCR4. After 1-day exposure to low oxygen, in vitro migration of MSCs in response to the fractalkine and SDF-1alpha increased in a dose dependent manner, while blocking antibodies for the chemokine receptors significantly decreased migration. Xenotypic grafting of cells from hypoxic cultures into early chick embryos demonstrated more efficient grafting of cells from hypoxic cultures compared to cells from normoxic cultures, and cells from hypoxic cultures generated a variety of cell types in host tissues. Other descriptions of hypoxic conditioning are described in the art. For example, cells can be cultured in hypoxic conditions or with gases that displace oxygen and/or cells can be treated with hypoxic mimetics.

Without wishing to be bound by theory, hypoxia has been demonstrated to induce expression of angiogenic genes in cells. For example, studies involving hypoxic preconditioning (HPC) of MSCs exposed to 0.5% oxygen for 24, 48, or 72 h before evaluating the expression of prosurvival, proangiogenic, and functional markers, such as hypoxia-inducible factor-1α, VEGF, phosphorylated Akt, survivin, p21, cytochrome c, caspase-3, caspase-7, CXCR4, and c-Met. MSCs exposed to 24-h hypoxia showed reduced apoptosis and had significantly higher levels of prosurvival, proangiogenic, and prodifferentiation proteins compared to MSCs exposed to 72-h hypoxia. Cells taken directly from a cryopreserved state did not respond as effectively to 24-h HPC as those cells cultured under normoxia before HPC. Cells cultured under normoxia before HPC showed decreased apoptosis and enhanced expression of connexin-43, cardiac myosin heavy chain, and CD31. The preconditioned cells were also able to differentiate into cardiovascular lineages. The results of the study suggest that MSCs cultured under normoxia before 24-h HPC are in a state of optimal expression of prosurvival, proangiogenic, and functional proteins that may increase subsequent survival after engraftment of the cells.

Thus, in some embodiments, regenerative fibroblast cells are exposed to 0.1% to 10% oxygen for a period of 30 minutes to 3 days, including 30 min to 2 days, 30 min to 1 day, 30 min to 12 hrs, 30 min to 6 hrs, 30 min to 1 hour, 1 hour to 3 days, 1 hour to 2 days, 1 hour to 1 day, 1-12 hours, 1-6 hrs, 12 hours to 3 days, 12 hours to 2 days, 12-24 hours, 1 day to 3 days, or 1-2 days, or 2-3 days. In some embodiments, regenerative fibroblast cells are exposed to 3% oxygen for 24 hours. In some embodiments, regenerative fibroblast cells are exposed to cobalt (II) chloride to chemically induce hypoxia. In some embodiments, regenerative fibroblast cells are exposed to cobalt (II) chloride for 1 to 48 hours, including 1-36, 1-24, 1-12, 12-48, 12-36, or 36-48 hours. In some embodiments, regenerative fibroblast cells are exposed to cobalt (II) chloride for 24 hours. In some embodiments, regenerative fibroblast cells are exposed to 1 μM-300 μM cobalt (II) chloride, including 1 μM-250 μM, 1 μM-200 μM, 1 μM-150 μM, 1 μM-100 μM, 1 μM-50 μM, 50 μM-300 μM, 50 μM-250 μM, 50 μM-200 μM, 50 μM-150 μM, 50 μM-100 μM, 100 μM-300 μM, 100 μM-250 μM, 100 μM-150 μM, 200 μM-300 μM, or 250 μM-300 μM. In some embodiments, regenerative fibroblast cells are exposed to 250 μM cobalt (II) chloride. In some embodiments, hypoxia induces an upregulation in HIF-1α, which is detected by expression of VEGF secretion. In some embodiments, hypoxia induces an upregulation of CXCR4 on fibroblast cells, which promotes homing of the cells to an SDF-1 gradient in inflamed areas.

Fibroblast cells used in the disclosed methods can undergo at least 25, 30, 35, or 40 doublings prior to reaching a senescent state. In some embodiments, fibroblasts are used prior to reaching senescence. Methods for deriving cells capable of doubling to reach 10¹⁴ cells or more are provided. In particular are those methods which derive cells that can double sufficiently to produce at least about 10¹⁴, 10¹⁵, 10¹⁶, or 10¹⁷ or more cells when seeded at from about 10³ to about 10⁶ cells/cm² in culture. In particular, these cell numbers are produced within 80, 70, or 60 days or less.

In some embodiments, the method optionally includes enriching populations of fibroblast cells. In one embodiment, cells are transfected with a polynucleotide vector containing a fibroblast-specific promoter operably linked to a reporter or selection gene. In some embodiments, the cell-specific promoter is an Oct-4, Nanog, Sox-9, GDF3, Rex-1, or Sox-2 promoter. In some embodiments, the method further includes the step of enriching the population for the regenerative fibroblast cells using expression of a reporter or selection gene. In some embodiments, the method further includes the step of enriching the population of the regenerative fibroblast cells by flow cytometry. In another embodiment, the method further comprises the steps of selecting fibroblast cells expressing CD105 and/or CD 117 and transfecting the fibroblast cells expressing CD105 and/or CD 117 with the NANOG gene.

In another embodiment, the method further includes the steps of contacting the fibroblast cells with a detectable compound that enters the cells, the compound being selectively detectable in proliferating and non-proliferating cells and enriching the population of cells for the proliferating cells. In some embodiments, the detectable compound is carboxyfluorescein diacetate, succinimidyl ester, and/or Aldefluor. In some embodiments, the fibroblast regenerative cell further comprises rhodamine-123 efflux activity. Without being bound by theory, cells possessing this property often express the multidrug resistance transport protein ABCG2 and are known for enhanced regenerative ability compared to cells which do not possess this efflux mechanism.

In some embodiments, fibroblast cells are obtained from a biopsy, and the donor providing the biopsy may be either the individual to be treated (autologous), the donor may be different from the individual to be treated (allogeneic). In some embodiments, the fibroblast cells are xenogenic. In some embodiments wherein allogeneic fibroblast cells are utilized for an individual, the fibroblast cells come from one or a plurality of donors. In some embodiments, steps are taken to protect allogeneic or xenogenic cells from immune mediated rejection by the recipient. Steps include encapsulation, co-administration of an immune suppressive agent, transfection of said cells with immune suppressory agent, or a combination thereof. In other embodiments, tolerance to the cells is induced through immunological means.

In some embodiments, fibroblasts are obtained from a source selected from the group consisting of dermal fibroblasts; placental fibroblasts; adipose fibroblasts; bone marrow fibroblasts; foreskin fibroblasts; umbilical cord fibroblasts; hair follicle derived fibroblasts; nail derived fibroblasts; endometrial derived fibroblasts; keloid derived fibroblasts; and a combination thereof.

In some embodiments, fibroblasts are fibroblasts isolated from placenta, umbilical cord, cord blood, peripheral blood, omentum, hair follicle, skin, bone marrow, adipose tissue, or Wharton's Jelly. In some embodiments, the fibroblasts are fibroblasts isolated from peripheral blood of a subject who has been exposed to conditions sufficient to stimulate fibroblasts from the subject to enter the peripheral blood. In another embodiment, fibroblast cells are mobilized by use of a mobilizing agent or therapy for treatment of ED. In some embodiments, the conditions and/or agents sufficient to stimulate fibroblasts from the subject to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof. In some embodiments, the mobilization therapy is selected from the group consisting of exercise, hyperbaric oxygen, autohemotherapy by ex vivo ozonation of peripheral blood, induction of SDF-1 secretion in an anatomical area outside of the bone marrow, or a combination thereof.

### III. Regenerative Fibroblast-Conditioned Media for Erectile Dysfunction Treatment

Certain aspects of the present disclosure relate to methods of reversing or substantially ameliorating the processes associated with erectile dysfunction through the therapeutic administration of concentrated media conditioned by regenerative fibroblast cells. In some embodiments, regenerative fibroblast cells are cultured in a growth medium to obtain conditioned media. In some embodiments, fibroblasts are cultured directly in tissue culture media including DMEM, EMEM, IMEM, or RPMI to produce fibroblast-conditioned media. In some embodiments, fibroblast-conditioned media is generated by culturing fibroblasts in hypoxic and/or hyperthermic conditions and/or with histone deacetylase inhibitors. In some embodiments, regenerative fibroblasts are also cultured alone or cultured in the presence of other cells to further upregulate production of growth factors in the conditioned media. Methods for generating conditioned media from fibroblasts are described herein.

In some embodiments, culture conditioned media is concentrated by filtering/desalting means known in the art. In one embodiment, filters with specific molecular weight cut-offs are utilized. In one embodiments, the filters select for molecular weights between 1 kDa and 50 kDa. In one embodiment, the cell culture supernatant is concentrated using means known in the art such as solid phase extraction using C18 cartridges (Mini-Speed C18-14%, S.P.E. Limited, Concord ON). C18 cartridges are used to adsorb small hydrophobic molecules from the stem or progenitor cell culture supernatant, and allows for the elimination of salts and other polar contaminants. The cartridges are prepared by washing with methanol, followed by washing with deionized-distilled water. In some embodiments, up to 100 ml of stem cell or progenitor cell supernatant may be passed through each of these specific cartridges before elution, though one of skill in the art would understand that larger cartridges may be used. After washing the cartridges, adsorbed material is eluted with methanol, evaporated under a stream of nitrogen, redissolved in a small volume of methanol, and stored at 4° C. Before testing the eluate for activity in vitro, the methanol is evaporated under nitrogen and replaced by culture medium. In other embodiments, different adsorption means known in the art are used to purify certain compounds from fibroblast cell supernatants.

In some embodiments, further purification and concentration is performed using gel filtration with a Bio-Gel P-2 column having a nominal exclusion limit of 1800 Da (Bio-Rad, Richmond Calif.). The column is washed and pre-swelled in 20 mM Tris-HCl buffer, pH 7.2, (Sigma) and degassed by gentle swirling under vacuum. Bio-Gel P-2 material is packed into a 1.5×0.54 cm glass column and equilibrated with 3 column volumes of the same buffer. Cell supernatant concentrates extracted by filtration are dissolved in 0.5 ml of 20 mM Tris buffer, pH 7.2, and run through the column. Fractions are collected from the column and analyzed for biological activity. In alternative embodiments, other purification, fractionation, and identification means known to one skilled in the art including anionic exchange chromatography, gas chromatography, high performance liquid chromatography, nuclear magnetic resonance, and mass spectrometry are used to prepare concentrated supernatants.

In some embodiments, active supernatant fractions are administered locally or systemically. The supernatant concentrated from fibroblast-conditioned media is assessed directly for biological activities or further purified. In one embodiment, the supernatants of fibroblast cultures are assessed for the ability to stimulate proteoglycan synthesis using an in vitro bioassay. In vitro bioassays allow for identification of the molecular weight fraction of the supernatant possessing biological activity and quantification of biological activity within the identified fractions. Bioassays testing the ability of the supernatant concentrates to stimulate regeneration of neuronal, smooth muscle, and/or endothelial cells are known in the art. Further, production of various proteins and biomarkers associated with regeneration of penile tissue is assessed by analysis of protein content using techniques including mass spectrometry, column chromatography, immune based assays such as enzyme linked immunosorbent assay (ELISA), immunohistochemistry, and flow cytometry.

### IV. Regenerative Fibroblast Cells for Erectile Dysfunction Treatment

Certain aspects of the present disclosure relate to the use of regenerative fibroblast cells for treatment or prevention of ED in an individual. Methods for generation of regenerative fibroblast cells are described elsewhere herein. In some embodiments, the disclosed methods comprise providing an effective amount of regenerative fibroblast cells to an individual sufficient to treat ED. Regenerative fibroblast cells may inhibit progression of pathological processes associated with ED and induce regenerative activity in the individual, thereby ameliorating or reversing ED in the individual. In some embodiments, a therapeutically effective amount of a composition comprising regenerative fibroblasts capable of inducing one or more biological activities is administered to an individual in need thereof. In some embodiments, the biological activities induced by the fibroblasts comprise inhibiting neuronal cell dysfunction, inhibiting cavernosal fibrosis, inhibiting smooth muscle degeneration, inhibiting biological pathways causative of ischemia, or a combination thereof.

In one embodiment, ED is treated by administration of reprogrammed fibroblasts. Reprogrammed fibroblasts results in fibroblasts having stem-cell like characteristics. Reprogrammed fibroblasts are selected from the group consisting of cells subsequent to a nuclear transfer, cells subsequent to a cytoplasmic transfer, cells treated with a DNA methyltransferase inhibitor, cells treated with a histone deacetylase inhibitor, cells treated with a GSK-3 inhibitor, cells induced to dedifferentiate by alteration of extracellular conditions, and cells exposed to various combinations of treatment conditions. In the case of cytoplasmic transfer, the cytoplasm of an undifferentiated cell, such as an inducible pluripotent cell, is transferred either by microinjection to the fibroblast, or by permeabilization of the fibroblast membrane. Subsequent to transfer of cytoplasm from an undifferentiated cell, the fibroblast takes on a phenotype of a more immature cell. By “immature,” it is meant that the fibroblast begins expressing markers of pluripotency, such as OCT-4, and/or NANOG, and/or SOX-2.

In another embodiment, ED is treated by in vitro or in vivo administration of fibroblast cells concurrently with one or more DNA demethylating agents selected from the group consisting of 5-azacytidine, psammaplin A, zebularine, and a combination thereof. In another embodiment, ED is treated by in vitro or in vivo administration of fibroblast cells concurrently with one or more DNA histone deacetylase inhibitors selected from the group consisting of valproic acid, trichostatin-A, trapoxin A, depsipeptide and a combination thereof.

In some embodiments, ED is treated by administration of fibroblast side population cells, wherein the side population cells are identified based on expression of the multidrug resistance transport protein (ABCG2) or the ability to efflux intracellular dyes such as rhodamine-123 and or Hoechst 33342. Without being bound to theory, cells possessing this property express stem-like genes and are known for enhanced regenerative ability compared to cells which do not possess this efflux mechanism. Fibroblast side population cells are derived from tissues including pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, or a combination thereof.

In another embodiment, ED is treated by administration of committed fibroblast progenitor cells selected from the group consisting of endothelial progenitor cells, neuronal progenitor cells, hematopoietic progenitor cells, and a combination thereof. Committed progenitor cells are those that have differentiated into a specific lineage or those which have been programmed to begin to differentiation. In some embodiments, the committed progenitor cells are committed endothelial progenitor cells purified from the bone marrow and/or peripheral blood. The committed endothelial progenitor cells are purified from the peripheral blood of a subject exposed to conditions sufficient to stimulate endothelial progenitor cells from the subject to enter the peripheral blood. In another aspect of the disclosure, endothelial progenitor cells are mobilized to enter the peripheral blood by use of a mobilizing agent or therapy for treatment of ED. In some embodiments, the one or more conditions and/or one or more agents sufficient to stimulate endothelial progenitor cells from the subject to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or combinations thereof. In some embodiments, the mobilization therapy is selected from the group consisting of exercise, hyperbaric oxygen, autohemotherapy by ex vivo ozonation of peripheral blood, induction of SDF-1 secretion in an anatomical area outside of the bone marrow, and a combination thereof.

In some embodiments, regenerative fibroblast cells are administered together with agents that increase endothelial cell activity. In other embodiments, regenerative fibroblasts are administered together with endothelial progenitor cells to increase endothelial cell activity. In still other embodiments, regenerative fibroblasts are administered together with endothelial cells to increase endothelial cell activity. The importance of the endothelial dysfunction in ED is exemplified by numerous assays that have demonstrated not only a correlation between endothelial dysfunction and ED, but also a correlation between reversion of ED and increased endothelial function. Studies demonstrating endothelial dysfunction include observations of reduced brachial flow dilation in ED patients, reduced reactive hyperemic response, impaired mean blood pressure and platelet aggregation responses to L-arginine, and reduced endothelial precursor cells in circulation. Interventions such as PDE5 inhibitors that successfully treat some forms of ED have been shown to increase both the number of circulating endothelial progenitors cells and the brachial flow-mediated dilation response. Interestingly, exercise, administration of statin drugs, and pregnancy have also been correlated with an increased number of circulating endothelial cells.

In one embodiment, one dose of CD34+ bone marrow derived stem cells at a concentration and/or frequency sufficient to induce endothelial responsiveness and nitric oxide production is administered to ED patients concurrently with fibroblasts. In another embodiment, several doses of CD34+ bone marrow derived stem cells at a concentration and/or frequency sufficient to induce endothelial responsiveness and nitric oxide production are administered to ED patients concurrently with fibroblasts. Without being bound to any particular theory or mechanism, induction of endothelial cell responsiveness may be due to fibroblasts acting to direct differentiation of fibroblast cells into endothelial cells, or via production of factors including IGF, EGF, and/or FGF-2 by the fibroblasts. In another embodiment, fibroblasts are stimulating differentiation of hematopoietic cells into endothelial cells, and/or endothelial precursor cells into endothelial cells. The dose and frequency of fibroblast administration can be determined based on endothelial function as assessed using assays such as the brachial flow-mediated dilation assay. In some embodiments, fibroblast cell infusions are tailored to allow restoration of erectile function in a patient-specific manner by quantifying endothelial responsiveness and the ability to produce NO in response to the infusions. Monitoring of function can be performed based on symptomology or on more quantitative scoring systems such as the Erectile Function Visual Analog Scale (EF-VAS) or the International Index of Erectile Function.

In another embodiment, fibroblast cells are administered in combination with testosterone therapy. In some embodiments, testosterone is administered at a concentration sufficient to induce smooth muscle cell growth in the areas associated with ED. Without wishing to be bound by theory, it is known that testosterone administration is useful for treatment of ED in patients suffering from hypogonadism, though testosterone-mediated improvement of erectile function in ED patients with basal levels of testosterone has not been clearly established. Testosterone treatment has, however, been shown to induce cell differentiation into a variety of muscle lineages, including smooth muscle tissue. Additionally, testosterone modulates endothelial function by increasing responsiveness to dilation stimuli and upregulating the ability of cells to generate and respond to NO.

Accordingly, in one embodiment, testosterone or testosterone derivatives or substitutes are administered to ED patients in combination with fibroblast cells. The goal of this therapy is to simultaneously increase the mass of smooth muscle cells in the penile area and provide endothelial cell progenitors to increase localized blood flow to the penile area. In some embodiments, testosterone is administered systemically, while in other embodiments, testosterone is administered locally. In some embodiments, patients with ED are treated with localized testosterone gel administered topically on the penile skin. In other embodiments, testosterone is administered by urethral suppository or by intracavernous injection. In some embodiments, testosterone is administered in the form of a precursor or a chemical modified form which possesses androgenic activity. Concentrations of testosterone to be administered vary on patient characteristics and route of administration. In some embodiments, concentrations of testosterone include 1-10 mg/day applied 1-4 times per day when applied topically on penile skin. In other embodiments, concentrations of testosterone range from 3-5 mg/day applied 1-3 times per day. In still other embodiments, the concentration of testosterone is approximately 4 mg/day applied twice per day.

In some embodiments, a therapeutically effective amount of one or more antioxidants are administered to a patient in need thereof. In some embodiments, the antioxidant is selected from the group consisting of ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, and a combination thereof. In some embodiments, one or more antioxidants are administered prior to administration of fibroblasts at a concentration sufficient to reduce oxidative stress from inhibiting the beneficial effects of the fibroblast cells on erectile dysfunction. In some antioxidant is administered concurrently with fibroblast cells in order to allow maximum cell beneficial function on erectile dysfunction. In some embodiments, the antioxidant is administered subsequent to fibroblast cell administration in order to allow the administered fibroblast cells to exert beneficial effects on erectile dysfunction.

Administration of fibroblast cells is local in some embodiments and systemic in others, with the preferred embodiment depending on individual patient characteristics. In one embodiment, fibroblasts are concentrated in an injection solution, which may be saline, mixtures of autologous plasma together with saline, or various concentrations of albumin with saline. In some embodiments, the pH of the injection solution is from about 6.4 to about 8.3, optimally 7.4. In some embodiments, excipients such as 4.5% mannitol, 0.9% sodium chloride, or pH buffers like sodium phosphate with art-known buffer solutions are used to bring the solution to isotonicity. In other embodiments, other pharmaceutically acceptable agents including but not limited to dextrose, boric acid, sodium tartrate, propylene glycol, polyols (such as mannitol and sorbitol), and other inorganic or organic solutes are used to bring the solution to isotonicity.

In some embodiments, fibroblast cell administration is performed through intravenous injection of autologous, allogeneic, or xenogeneic fibroblast cells. Concentration of fibroblast cells administered ranges from 1×10⁶-10×10⁷ cells in some embodiments, to between 1×10⁶-5×10⁷ cells in other embodiments and between 1×10⁶-1×10⁷ cells in still other embodiments. In some embodiments, fibroblast cell administration is performed as a single event, while in other embodiments, administration is performed in multiple cycles, with the preferred embodiment depending on the functional result obtained and individual patient characteristics. In other embodiments, the cells are administered in combination with other agents known to increase erectile function including but not limited to inhibitors of PDE-5, PGE-1, papaverine, promorphine, other known vasodilators, or a combination thereof. Efficacy of fibroblast cell therapy is quantified using standard scales of sexual and erectile function and more objective techniques such as Doppler ultrasonography, angiography, or nerve-mediated erectile stimulation.

### V. Kits of the Disclosure

Any of the cellular and/or non-cellular compositions described herein or similar thereto may be comprised in a kit. In a non-limiting example, one or more reagents for use in methods for preparing fibroblasts or derivatives thereof may be comprised in a kit. Such reagents may include cells, vectors, one or more growth factors, vector(s), one or more costimulatory factors, media, enzymes, buffers, nucleotides, salts, primers, compounds, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate particular embodiments of the invention. It should be appreciated by those of skill in the art that the techniques disclosed in the examples that follow represent techniques discovered by the inventors to function well in the practice of the methods of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### The Unexpected Finding of Increased Sexual Potency in Critical Limb Ischemia Patients Treated With Fibroblasts

Twenty-five patients suffering from Fountain III and IV critical limb ischemia were selected for fibroblast administration based on non-responsiveness to medical interventions and ineligibility for surgical intervention. Inclusion into the study required measurable haemodynamic deficits included resting ankle-brachial pressure index (ABI) less than 0.6 in the affected limb on two consecutive examinations done at least 1 week apart. Patients with poorly controlled diabetes mellitus (HbA1c >6.5% and proliferative retinopathy) or with evidence of malignant disorder during the past 5 years were excluded due to the potential of bone marrow cells to stimulate angiogenesis. Written informed consent was obtained from all patients. Ethics committees of participating universities approved the protocol.

Fibroblasts were injected into patients either in the gluteus maximus muscles (17 patients) or in the gastrocnemius muscle (8 patients) at concentrations ranging from 5×10⁶⁻³×10⁷. Two months post cellular administration, increased sexual potency and ability to perform sexually was spontaneously reported in 16 of the 17 patients injected into the gluteus maximus muscles and in 1 of the 8 patients injected into the gastrocnemius muscle.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
U.S. Pat. No. 5,250,534
U.S. Pat. No. 5,773,020
U.S. Pat. No. 4,127,118
U.S. Pat. No. 5,518,499
U.S. Pat. No. 3,943,246
U.S. Pat. No. 4,530,920
U.S. Pat. No. 4,139,617
U.S. Pat. No. 5,541,211
U.S. Pat. No. 5,770,606
U.S. Pat. No. 4,801,587
U.S. Pat. No. 5,065,744

What is claimed is:

1. A method of treating or preventing erectile dysfunction in an individual, comprising the step of administering a therapeutically effective amount of a composition comprising fibroblasts and/or conditioned media therefrom to an individual in need thereof.

2. The method of claim 1, wherein the fibroblasts comprise regenerative fibroblasts.

3. The method of claim 1, wherein the fibroblast cells were cultured under conditions sufficient to differentiate the fibroblasts into regenerative fibroblast cells.

4. The method of claims 2 or 3, wherein the regenerative fibroblast cells comprise one or more of the following biological activities:
(a) induction of angiogenesis;
(b) prevention of tissue atrophy;
(c) regeneration of functional tissue;
(d) inhibition of neuronal cell dysfunction;
(e) inhibition of cavernosal fibrosis;
(f) inhibition of smooth muscle degeneration; and
(g) inhibition of one or more biological pathways causative of ischemia.

5. The method of any one of claims 2-4, wherein the regenerative fibroblast cells are cultured under conditions sufficient to enhance the ability of the regenerative fibroblast cells to induce angiogenesis, prevent tissue atrophy, regenerate functional tissue, inhibit neuronal cell dysfunction, inhibit cavernosal fibrosis, inhibit smooth muscle degeneration, inhibit biological pathways causative of ischemia, or a combination thereof.

6. The method of any one of claims 3-5, wherein the conditions comprise hypoxia.

7. The method of claim 6, wherein the hypoxic conditions comprise from 0.1% oxygen to 10% oxygen for a period of 30 minutes to 3 days.

8. The method of claim 7 wherein the hypoxic conditions comprise 3% oxygen for 24 hours.

9. The method of claim 6, wherein hypoxic conditions are chemically induced.

10. The method of claim 9, wherein chemical induction of hypoxia comprises culture in cobalt (II) chloride.

11. The method of claim 10, wherein fibroblast cells are cultured with 1 μM-300 μM cobalt (II) chloride.

12. The method of claim 11, wherein the fibroblast cells are incubated with 250 μM of cobalt (II) chloride.

13. The method of claims 9-12, wherein the fibroblast cells are further cultured for 1-48 hours.

14. The method of claims 9-13, wherein the fibroblast cells are cultured for a time period of 24 hours.

15. The method of claims 6-14, wherein the hypoxic conditions induce upregulation of HIF-1α.

16. The method of claim 15, wherein expression of HIF-1α is detected by expression of VEGF secretion.

17. The method of claim 6-16, wherein the hypoxic conditions induce upregulation of CXCR4 on the fibroblast cells.

18. The method of claim 17, wherein upregulation of CXCR4 promotes homing of the fibroblast cells to an SDF-1 gradient.

19. The method of any one of claims 3-18, wherein the conditions further comprise treatment of the regenerative fibroblast cells with one or more growth factors, one or more differentiation factors, one or more dedifferentiation factors, or a combination thereof.

20. The method of any one of claims 2-19, wherein the regenerative fibroblast cells express one or more markers selected from the group consisting of Oct-4, Nanog, Sox-2, KLF4, c-Myc, Rex-1, GDF-3, LIF receptor, CD105, CD117, CD344, Stella, and a combination thereof.

21. The method of any one of claims 2-20, wherein the regenerative fibroblast cells do not express one or more cell surface proteins selected from the group consisting of MHC class I, MHC class II, CD45, CD13, CD49c, CD66b, CD73, CD105, CD90, and a combination thereof.

22. The method of any one of claims 2-21, wherein the regenerative fibroblast cells have enhanced GDF-11 expression compared to a control or standard.

23. The method of any one of claims 1-22, wherein the fibroblast cells are, or are derived from, fibroblasts isolated from umbilical cord, skin, cord blood, adipose tissue, hair follicle, omentum, bone marrow, peripheral blood, Wharton's Jelly, or a combination thereof.

24. The method of any one of claims 1-23, wherein the fibroblast cells are obtained from dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof.

25. The method of any one of claims 1-24, wherein the fibroblast cells are autologous, allogeneic, or xenogeneic to the recipient.

26. The method of any one of claims 1-25, wherein the fibroblast cells are purified from bone marrow.

27. The method of any one of claims 1-25, wherein the fibroblast cells are purified from peripheral blood.

28. The method of any one of claims 2-27, wherein the regenerative fibroblast cells are isolated from peripheral blood of an individual who has been exposed to one or more conditions and/or one or more therapies sufficient to stimulate regenerative fibroblast cells from the individual to enter the peripheral blood of the individual.

29. The method of claim 28, wherein the conditions sufficient to stimulate regenerative fibroblast cells from the individual to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof.

30. The method of claim 28, wherein the therapies sufficient to stimulate regenerative fibroblast cells from the individual to enter the peripheral blood comprise therapies including exercise, hyperbaric oxygen, autohemotherapy by ex vivo ozonation of peripheral blood, induction of SDF-1 secretion in an anatomical area outside of the bone marrow, or a combination thereof.

31. The method of any one of claims 2-30, wherein the regenerative fibroblast cells are comprised of an enriched population of regenerative fibroblast cells.

32. The method of claim 31, wherein enrichment is achieved by:
(a) transfecting the cells with a vector comprising a fibroblast-specific promoter operably linked to a reporter or selection gene, wherein the reporter or selection gene is expressed, and
(b) enriching the population of cells for cells expressing the reporter or selection gene.

33. The method of claim 31, wherein enrichment is achieved by:
(a) treating the cells with a detectable compound, wherein the detectable compound is selectively detectable in proliferating and non-proliferating cells, and
(b) enriching the population of cells for proliferating cells.

34. The method of claim 33, wherein the detectable compound is selected from a group comprising carboxyfluorescein diacetate, succinimidyl ester, and Aldefluor.

35. The method of any one of claims 2-34, wherein the regenerative fibroblast cells are reprogrammed fibroblasts.

36. The method of claim 35, wherein the reprogrammed fibroblasts are selected from the group consisting of cells subsequent to a nuclear transfer, cells subsequent to a cytoplasmic transfer, cells treated with one or more DNA methyltransferase inhibitors, cells treated with one or more histone deacetylase inhibitors, cells treated with one or more GSK-3 inhibitors, cells induced to dedifferentiate by alteration of one or more extracellular conditions, and cells exposed to various combinations of treatment conditions.

37. The method of claim 36, wherein the DNA methyltransferase inhibitor is selected from the group consisting of 5-azacytidine, psammaplin A, zebularine, and a combination thereof.

38. The method of claim 36, wherein the DNA histone deacetylase inhibitor is selected from the group consisting of valproic acid, trichostatin-A, trapoxin A, depsipeptide, and a combination thereof.

39. The method of any one of claims 2-38, wherein the regenerative fibroblast cells are fibroblasts isolated as side population cells.

40. The method of claim 39, wherein the fibroblasts isolated as side population cells are identified based on expression of the multidrug resistance transport protein (ABCG2).

41. The method of claim 39, wherein the fibroblasts isolated as side population cells are identified based on the ability to efflux intracellular dyes.

42. The method of any one of claims 39-41, wherein the side population cells are derived from tissues selected from the group consisting of pancreatic tissue, liver tissue, smooth muscle tissue, striated muscle tissue, cardiac muscle tissue, bone tissue, bone marrow tissue, bone spongy tissue, cartilage tissue, liver tissue, pancreas tissue, pancreatic ductal tissue, spleen tissue, thymus tissue, Peyer's patch tissue, lymph nodes tissue, thyroid tissue, epidermis tissue, dermis tissue, subcutaneous tissue, heart tissue, lung tissue, vascular tissue, endothelial tissue, blood cells, bladder tissue, kidney tissue, digestive tract tissue, esophagus tissue, stomach tissue, small intestine tissue, large intestine tissue, adipose tissue, uterus tissue, eye tissue, lung tissue, testicular tissue, ovarian tissue, prostate tissue, connective tissue, endocrine tissue, mesentery tissue, and a combination thereof.

43. The method of any one of claims 1-42, wherein a committed fibroblast progenitor cell population is administered together with the fibroblast cells.

44. The method of claim 43, wherein the committed fibroblast progenitor cells are selected from the group consisting of committed endothelial progenitor cells, committed neuronal progenitor cells, committed hematopoietic progenitor cells, and a combination thereof.

45. The method of claim 44, wherein a committed endothelial progenitor cell population is administered in combination with the fibroblast cells.

46. The method of claim 45, wherein the committed endothelial progenitor cells express one or more markers selected from the group consisting of CD31, CD34, AC133, CD146, flk1, and a combination thereof.

47. The method of any one of claims 45-46, wherein the committed endothelial progenitor cells are autologous, allogeneic, or xenogeneic to the recipient.

48. The method of any one of claims 45-47, wherein the committed endothelial progenitor cells are obtained from bone marrow.

49. The method of any one of claims 45-47, wherein the committed endothelial progenitor cells are obtained from peripheral blood.

50. The method of any one of claim 45-47 or 49, wherein the committed endothelial progenitor cells are isolated from peripheral blood of an individual who has been exposed to one or more conditions and/or one or more therapies sufficient to stimulate endothelial progenitor cells from the individual to enter the peripheral blood of the individual.

51. The method of claim 50, wherein the one or more conditions sufficient to stimulate committed endothelial progenitor cells from the individual to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof.

52. The method of claim 50, wherein the one or more therapies sufficient to stimulate committed endothelial progenitor cells from the individual to enter the peripheral blood comprise therapies including exercise, hyperbaric oxygen, autohemotherapy by ex vivo ozonation of peripheral blood, induction of SDF-1 secretion in an anatomical area outside of the bone marrow, or a combination thereof.

53. The method of any one of claims 1-52, wherein testosterone is also administered to the individual.

54. The method of claim 53, wherein the concentration of testosterone is sufficient to induce smooth muscle cell growth.

55. The method of claim 53 or 54, wherein the concentration of testosterone is sufficient to induce migration of endothelial progenitor cells to the penis and/or upstream of the penis.

56. The method of any one of claims 53-55, wherein the testosterone is administered systemically or locally to the individual.

57. The method of claim 56, wherein local administration of testosterone is by topical application, urethral suppository, and/or intracavernous injection.

58. The method of any one of claims 1-57, wherein a therapeutically effective amount of one or more antioxidants are administered to the individual.

59. The method of claim 58, wherein the one or more antioxidants are selected from the group consisting of ascorbic acid and derivatives thereof, alpha tocopherol and derivatives thereof, rutin, quercetin, hesperedin, lycopene, resveratrol, tetrahydrocurcumin, rosmarinic acid, Ellagic acid, chlorogenic acid, oleuropein, alpha-lipoic acid, glutathione, polyphenols, pycnogenol, and a combination thereof.

60. The method of claim 58 or 59, wherein the antioxidant is administered prior to administration of the fibroblast cells at a concentration sufficient to reduce oxidative stress.

61. The method of claim 58 or 59, wherein the antioxidant is administered concurrently with the fibroblast cells at a concentration sufficient to reduce oxidative stress.

62. The method of claim 58 or 59, wherein the antioxidant is administered subsequent to the fibroblast cells at a concentration sufficient to reduce oxidative stress.

63. The method of any one of claims 1-62, wherein the erectile dysfunction comprises vascular insufficiency.

64. The method of any one of claims 1-62, wherein the erectile dysfunction comprises neuronal dysfunction.

65. The method of any one of claims 1-62, wherein the erectile dysfunction comprises fibrosis of the corpora cavernous, the corpus spongiosum, or a combination thereof.

66. The method of any one of claims 1-62, wherein the erectile dysfunction is associated with injury.

67. The method of claim 66, wherein the injury is traumatic.

68. The method of claim 66, wherein the injury is surgical.

69. The method of claim 66, wherein the injury is atherosclerotic.

70. The method of claim 66, wherein the injury is due to age-associated degeneration of neurons, smooth muscle, or a combination thereof.

71. The method of claim 70, wherein structural elements of the neurons, smooth muscle, or a combination thereof degenerate.

72. The method of claim 71, wherein structural elements include cellular morphology, cytoskeleton shape, and subcellular organelles.

73. The method of claims 70-72, wherein functional elements of the neurons, smooth muscle, or a combination thereof degenerate.

74. The method of claim 73, wherein functional elements include the ability of neurons to produce and respond to neurotransmitters and/or the ability of in the cause of smooth muscle to contract upon receiving contractile signals.

75. The method of any one of claims 1-74, further defined as administering to the individual a therapeutically effective amount of a composition comprising regenerative fibroblast-conditioned media.

76. The method of claim 75, wherein regenerative fibroblast cells are cultured under conditions sufficient to upregulate production of one or more growth factors in the regenerative fibroblast-conditioned media.

77. The method of claim 76, wherein the conditions comprise hypoxia, hyperthermia, treatment with histone deacetylase inhibitors, or a combination thereof.

78. The method of claims 75-77, wherein the regenerative fibroblast-conditioned media is concentrated.

79. The method of claims 75-78, wherein the regenerative fibroblast-conditioned media is administered locally or systemically to the individual.

80. A method for generating regenerative fibroblast cells from fibroblasts, comprising subjecting fibroblasts to one or more conditions sufficient to generate regenerative fibroblast cells from the fibroblasts.

81. The method of claim 80, wherein the conditions sufficient to generate regenerative fibroblast cells from fibroblasts comprise conditions sufficient to enhance the ability of the regenerative fibroblast cells to induce angiogenesis, prevent tissue atrophy, regenerate functional tissue, inhibit neuronal cell dysfunction, inhibit cavernosal fibrosis, inhibit smooth muscle degeneration, inhibit biological pathways causative of ischemia, or a combination thereof.

82. The method of claim 80 or 81, wherein the conditions include hypoxia.

83. The method of claim 82, wherein the hypoxic conditions comprise from 0.1% oxygen to 10% oxygen for a period of 30 minutes to 3 days.

84. The method of claim 83 wherein the hypoxic conditions comprise 3% oxygen for 24 hours.

85. The method of claim 82, wherein hypoxic conditions are chemically induced.

86. The method of claim 85, wherein chemical induction of hypoxia comprises culture in cobalt (II) chloride.

87. The method of claim 86, wherein fibroblast cells are cultured with 1 μM-300 μM cobalt (II) chloride.

88. The method of claim 87, wherein the fibroblast cells are incubated with 250 μM of cobalt (II) chloride.

89. The method of claims 85-88, wherein the fibroblast cells are further cultured for 1-48 hours.

90. The method of claims 85-89, wherein the fibroblast cells are cultured for a time period of 24 hours.

91. The method of claims 82-90, wherein the hypoxic conditions induce upregulation of HIF-1α.

92. The method of claim 91, wherein expression of HIF-1α is detected by expression of VEGF secretion.

93. The method of claim 82-92, wherein the hypoxic conditions induce upregulation of CXCR4 on the fibroblast cells.

94. The method of claim 93, wherein upregulation of CXCR4 promotes homing of the fibroblast cells to an SDF-1 gradient.

95. The method of any one of claims 80-94, wherein the regenerative fibroblast cells are treated with one or more growth factors, one or more differentiation factors, one or more dedifferentiation factors, or a combination thereof.

96. The method of any one of claims 80-95, wherein the regenerative fibroblast cells express one or more markers comprising Oct-4, Nanog, Sox-2, KLF4, c-Myc, Rex-1, GDF-3, LIF receptor, CD105, CD117, CD344, Stella, or a combination thereof.

97. The method of any one of claims 80-96, wherein the regenerative fibroblast cells do not express one or more cell surface proteins comprising MHC class I, MHC class II, CD45, CD13, CD49c, CD66b, CD73, CD105, CD90, or a combination thereof.

98. The method of any one of claims 80-97, wherein the regenerative fibroblast cells comprise enhanced GDF-11 expression compared to a control or standard.

99. The method of any one of claims 80-98, wherein the regenerative fibroblast cells are fibroblasts isolated from umbilical cord, skin, cord blood, adipose tissue, hair follicle, omentum, bone marrow, peripheral blood, Wharton's Jelly, or a combination thereof.

100. The method of any one of claims 80-99, wherein the regenerative fibroblast cells are obtained from dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof.

101. The method of any one of claims 80-100, wherein the regenerative fibroblast cells are autologous, allogeneic, or xenogeneic with respect to the individual.

102. The method of any one of claims 80-101, wherein the regenerative fibroblast cells are obtained from bone marrow.

103. The method of any one of claims 80-101, wherein the regenerative fibroblast cells are obtained from peripheral blood.

104. The method of any one of claim 80-101 or 103, wherein the regenerative fibroblast cells are isolated from peripheral blood of an individual who has been exposed to one or more conditions or one or more therapies sufficient to stimulate regenerative fibroblast cells from the individual to enter the peripheral blood.

105. The method of claim 104, wherein the conditions sufficient to stimulate regenerative fibroblast cells from the individual to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof.

106. The method of claim 104, wherein the therapies sufficient to stimulate regenerative fibroblast cells from the individual to enter the peripheral blood comprise therapies including exercise, hyperbaric oxygen, autohemotherapy by ex vivo ozonation of peripheral blood, induction of SDF-1 secretion in an anatomical area outside of the bone marrow, or a combination thereof.

107. A regenerative fibroblast cell wherein the regenerative fibroblast cell is derived from a fibroblast cultured under one or more conditions sufficient to enhance the ability of the regenerative fibroblast cells to induce angiogenesis, prevent tissue atrophy, regenerate functional tissue, inhibit neuronal cell dysfunction, inhibit cavernosal fibrosis, inhibit smooth muscle degeneration, inhibit biological pathways causative of ischemia, or a combination thereof.

108. The regenerative fibroblast cell of claim 107, wherein the conditions comprise hypoxia.

109. The method of claim 108, wherein the hypoxic conditions comprise from 0.1% oxygen to 10% oxygen for a period of 30 minutes to 3 days.

110. The method of claim 109 wherein the hypoxic conditions comprise 3% oxygen for 24 hours.

111. The method of claim 108, wherein hypoxic conditions are chemically induced.

112. The method of claim 111, wherein chemical induction of hypoxia comprises culture in cobalt (II) chloride.

113. The method of claim 112, wherein fibroblast cells are cultured with 1 μM-300 μM cobalt (II) chloride.

114. The method of claim 113, wherein the fibroblast cells are incubated with 250 μM of cobalt (II) chloride.

115. The method of claims 85-88, wherein the fibroblast cells are further cultured for 1-48 hours.

116. The method of claims 111-115, wherein the fibroblast cells are cultured for a time period of 24 hours.

117. The method of claims 108-116, wherein the hypoxic conditions induce upregulation of HIF-1α.

118. The method of claim 117, wherein expression of HIF-1α is detected by expression of VEGF secretion.

119. The method of claim 108-118, wherein the hypoxic conditions induce upregulation of CXCR4 on the fibroblast cells.

120. The method of claim 119, wherein upregulation of CXCR4 promotes homing of the fibroblast cells to an SDF-1 gradient.

121. The regenerative fibroblast cell of any one of claims 107-120, wherein the regenerative fibroblast cell is treated with one or more growth factors, one or more differentiation factors, one or more dedifferentiation factors, or a combination thereof.

122. The regenerative fibroblast cell of any one of claims 107-121, wherein the regenerative fibroblast cell expresses one or more markers comprising Oct-4, Nanog, Sox-2, KLF4, c-Myc, Rex-1, GDF-3, LIF receptor, CD105, CD117, CD344, Stella, or a combination thereof.

123. The regenerative fibroblast cell of any one of claims 107-122, wherein the regenerative fibroblast cell does not express one or more cell surface proteins comprising MHC class I, MHC class II, CD45, CD13, CD49c, CD66b, CD73, CD105, CD90, or a combination thereof.

124. The regenerative fibroblast cell of any one of claims 107-123, wherein the regenerative fibroblast cell has enhanced GDF-11 expression compared to a control or standard.

125. The regenerative fibroblast cell of any one of claims 107-124, wherein the regenerative fibroblast cell is a fibroblast isolated from umbilical cord, skin, cord blood, adipose tissue, hair follicle, omentum, bone marrow, peripheral blood, Wharton's Jelly, or a combination thereof.

126. The regenerative fibroblast cell of any one of claims 107-125, wherein the regenerative fibroblast cell is obtained from dermal fibroblasts, placental fibroblasts, adipose fibroblasts, bone marrow fibroblasts, foreskin fibroblasts, umbilical cord fibroblasts, hair follicle derived fibroblasts, nail derived fibroblasts, endometrial derived fibroblasts, keloid derived fibroblasts, or a combination thereof.

127. The regenerative fibroblast cell of any one of claims 107-126, wherein the regenerative fibroblast cell is autologous, allogeneic, or xenogeneic with respect to the individual.

128. The regenerative fibroblast cell of any one of claims 107-127, wherein the regenerative fibroblast cell is obtained from bone marrow.

129. The regenerative fibroblast cell of any one of claims 107-127, wherein the regenerative fibroblast cell is obtained from peripheral blood.

130. The regenerative fibroblast cell of any one of claim 107-127 or 129, wherein the regenerative fibroblast cell is isolated from peripheral blood of an individual who has been exposed to one or more conditions and/or one or more therapies sufficient to stimulate regenerative fibroblast cells from the individual to enter the peripheral blood of the individual.

131. The regenerative fibroblast cell of claim 130, wherein the one or more conditions sufficient to stimulate regenerative fibroblast cells from the individual to enter the peripheral blood comprise administration of G-CSF, M-CSF, GM-CSF, 5-FU, IL-1, IL-3, kit-L, VEGF, Flt-3 ligand, PDGF, EGF, FGF-1, FGF-2, TPO, IL-11, IGF-1, MGDF, NGF, HMG CoA reductase inhibitors, small molecule antagonists of SDF-1, or a combination thereof.

132. The regenerative fibroblast cell of claim 130, wherein the one or more therapies sufficient to stimulate regenerative fibroblast cells from an individual to enter the peripheral blood comprise therapies including exercise, hyperbaric oxygen, autohemotherapy by ex vivo ozonation of peripheral blood, induction of SDF-1 secretion in an anatomical area outside of the bone marrow, or a combination thereof.

133. A regenerative fibroblast-conditioned media for erectile dysfunction treatment wherein the media is derived from regenerative fibroblast cells cultured under conditions sufficient to upregulate production of one or more growth factors.

134. The regenerative fibroblast-conditioned media of claim 133, wherein the conditions comprise hypoxia, hyperthermia, treatment with histone deacetylase inhibitors, or a combination thereof.

135. The regenerative fibroblast-conditioned media of claim 133 or 134, wherein the regenerative fibroblast-conditioned media is concentrated.

136. The regenerative fibroblast-conditioned media of claim 133-135, wherein the regenerative fibroblast-conditioned media is administered locally or systemically to an individual.
